(19) United States
(12) Patent Application Publication
(10) Pub. No.: US 2007/0172841 A1
(43) Pub. Date: Jul. 26, 2007

(54) Probe/target stabilization with add-in oligo

(76) Inventor: Hui Wang, Palo Alto, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/340,338

(22) Filed: Jan. 25, 2006

(51) Int. Cl. C12Q 1/68; C12M 3/00

(52) U.S. Cl. 435/6; 435/287.2

(57) ABSTRACT

The invention relates to analyzing a sample containing small RNAs. In exemplary embodiments, the sample is contacted with an array in the presence of an add-in oligo. In typical embodiments, probes on the array include regions that are complementary to small RNAs and regions that are complementary to the add-in oligo. The array is then interrogated to obtain information about small RNA in the sample. Arrays and kits in accordance with the invention are also described.

### RELATED APPLICATIONS

Related subject matter is disclosed copending U.S. patent application Ser. No. 11/173,693 filed on Jul. 1, 2005 by Wang entitled “Nucleic Acid Probes for Analysis of Small RNAs and Other Polynucleotides”; the copending U.S. patent application Ser. No. 11/256,229 filed on Oct. 21, 2005 by Wang et al. entitled “Analysis of microRNA” and designated attorney docket no. 10051514-1; and the copending U.S. patent application Ser. No. 11/264,788 filed on Oct. 31, 2005 by Wang entitled “Probe/Target Stabilization with Add-In Oligo” and designated attorney docket no. 10051644-1.

### FIELD OF THE INVENTION

The invention relates generally to methods of biochemical analysis. More specifically, the invention relates to a method of analyzing a sample containing polynucleotides.

### BACKGROUND OF THE INVENTION

Since the discovery of the biological activity of short interfering RNAs (siRNAs) over a decade ago, so called “small RNAs” (i.e., short non-coding regulatory RNAs that have a defined sequence) have become a subject of intense interest in the research community. See Novina et al., Nature 430: 161-164 (2004). Exemplary small RNAs include siRNAs, microRNAs (miRNAs), tiny non-coding RNAs (tncRNAs) and small modulatory RNAs (smRNAs), as well as many others.

Although the exact biological functions of most small RNAs remain a mystery, it is clear that they are abundant in plants and animals, with up to tens of thousands of copies per cell. For example, to date, over 78 Drosophila microRNA species and 300 human microRNA species have been identified. The levels of the individual species of small RNA, in particular microRNA species, appear to vary according to the developmental stage and type of tissue being examined. It is thought that the levels of particular small RNAs may be correlated with particular phenotypes, as well as with the levels of particular messenger RNAs and proteins. Further, viral microRNAs have been identified, and their presence has been linked to viral latency (see Pfeffer et al., Science, 304: 734-736 (2004)).

The sequences of several hundred miRNAs from a variety of different species, including humans, may be found at the microRNA registry (Griffiths-Jones, Nucl. Acids Res. 2004 32:D109-D111), as found at the world-wide website of the Sanger Institute (Cambridge, UK) (which may be accessed by typing “www” followed by “.sanger.ac.uk/cgi-bin/Rfam/mirna/browse.pl” into the address bar of a typical internet browser). The sequences of all of the microRNAs deposited at the microRNA registry, including more than 300 microRNA sequences from humans (see Lagos-Quintana et al, Science 294:853-858(2001); Grad et al, Mol Cell 11:1253-1263(2003); Mourelatos et al, Genes Dev 16:720-728(2002); Lagos-Quintana et al, Curr Biol 12:735-739(2002); Lagos-Quintana et al, RNA 9:175-179(2003); Dostie et al, RNA 9:180-186(2003); Lim et al, Science 299:1540(2003); Houbaviy et al, Dev Cell 5:351-358(2003); Michael et al, Mol Cancer Res 1:882-891(2003); Kim et al, Proc Natl Acad Sci USA 101:360-365(2004); Suh et al, Dev Biol 270:488-498(2004); Kasashima et al, Biochem Biophys Res Commun 322:403-410(2004); and Xie et al, Nature 434:338-345(2005)), are incorporated herein by reference. MicroRNAs (miRNAs) are a class of single stranded RNAs of approximately 19-25 nt (nucleotides) in length.

Thus, analysis of miRNA may be of great importance, for example as a research or diagnostic tool. Analytic methods employing polynucleotide arrays have been used for investigating small RNAs, e.g. miRNAs have become a subject of investigation with microarray analysis. See, e.g., Liu et al., Proc. Nat'l Acad. Sci. USA, 101: 9740-9744 (2004); Thomson et al., Nature Methods, 1:1-7 (2004); and Babak et al., RNA, 10: 1813-1819 (2004). A considerable amount of effort is currently being put into developing array platforms to facilitate the analysis of small RNAs, particularly microRNAs. Polynucleotide arrays (such as DNA or RNA arrays) typically include regions of usually different sequence polynucleotides (“capture agents”) arranged in a predetermined configuration on an array support. The arrays are “addressable” in that these regions (sometimes referenced as “array features”) have different predetermined locations (“addresses”) on the array support. The polynucleotide arrays typically are fabricated on planar array supports either by depositing previously obtained polynucleotides onto the array support in a site specific fashion or by site specific in situ synthesis of the polynucleotides upon the array support. After depositing the polynucleotide capture agents onto the array support, the array support is typically processed (e.g., washed and blocked for example) and stored prior to use.

In use, an array is contacted with a sample (e.g. a labeled sample) containing analytes (typically, but not necessarily, other polynucleotides) under conditions that promote specific binding of the analytes in the sample to one or more of the capture agents present on the array. Thus, the arrays, when exposed to a sample, will undergo a binding reaction with the sample and exhibit an observed binding pattern. This binding pattern can be detected upon interrogating the array. For example all target polynucleotides (for example, DNA) in the sample can be labeled with a suitable label (such as a fluorescent compound), and the label then can be accurately observed (such as by observing the fluorescence pattern) on the array after exposure of the array to the sample. Assuming that the different sequence polynucleotides were correctly deposited in accordance with the predetermined configuration, then the observed binding pattern will be indicative of the presence and/or concentration of one or more components of the sample. Techniques for scanning arrays are described, for example, in U.S. Pat. No. 5,763,870 and U.S. Pat. No. 5,945,679. Still other techniques useful for observing an array are described in U.S. Pat. No. 5,721,435.

Straightforward and reliable methods for simultaneously analyzing several constituents of a complex RNA sample are extremely desirable. While current methods of preparing and analyzing RNA samples are quite useful, there is a continuing need for development of such methods.

### SUMMARY OF THE INVENTION

The invention thus relates to novel methods of performing an array analysis of an RNA sample. In certain embodiments, the invention provides a method of analyzing a sample containing small RNAs. The sample is contacted with an array in the presence of an add-in oligo under conditions sufficient to provide for binding to the array. The array has a set of probes bound to an array support. Each probe of the set has a target complementary region bound to the array support and an add-in oligo complementary region bound to the target complementary region. Thus, the add-in oligo complementary region is bound to the array support via the target complementary region. The array is then interrogated to obtain information about small RNAs in the sample. Arrays and kits in accordance with the present invention are also described.

Additional objects, advantages, and novel features of this invention are set forth in part in the description follows and in part will become apparent to those skilled in the art upon examination of the following specifications or may be learned by the practice of the invention. The objects and advantages of the invention may be realized and attained by means of the instruments, combinations, compositions and methods particularly pointed out herein and in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1 schematically illustrates an embodiment of an array in accordance with the present invention;

FIG. 2 schematically illustrates an embodiment in which an array is contacted with the sample in the presence of an add-in oligo under conditions sufficient to provide for binding to the array; and

FIG. 3 schematically illustrates various embodiments of probes on an array support.

To facilitate understanding, identical reference numerals have been used, where practical, to designate corresponding elements that are common to the Figures. The Figure components are broadly illustrative and are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “an oligonucleotide” includes a plurality of oligonucleotides. Similarly, reference to “an RNA” includes a plurality of different identity (sequence) RNA species.

Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. In this specification and in the claims that follow, reference will be made to a number of terms that shall be defined to have the following meanings unless a contrary intention is apparent.

“Optional” or “optionally” means that the subsequently described circumstance may or may not occur, so that the description includes instances where the circumstance occurs and instances where it does not. For example, if a step of a process is optional, it means that the step may or may not be performed, and, thus, the description includes embodiments wherein the step is performed and embodiments wherein the step is not performed (i.e. it is omitted). As another example, if a portion of a polynucleotide sequence is optional, it means that the portion may or may not be a part of the polynucleotide sequence, and, thus, the description includes embodiments wherein the portion is present and embodiments wherein the portion is not present (i.e. it is omitted).

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein; further e.g., oligomers of Locked Nucleoside Analogues (LNAs) as described in U.S. Pat. No. 6,794,499; further e.g., polynucleotides having non-natural nucleotides that provide for reduced secondary structure such as UNA described in U.S. patent application Ser. No. 09/358,141 filed Jul. 20, 1999) which can hybridize with naturally occurring nucleic acids in a sequence specific manner similar to that of two naturally occurring nucleic acids, e.g., can participate in Watson-Crick base pairing interactions. An “oligonucleotide” is a molecule containing from 2 to about 100 nucleotides. The terms “nucleoside”, “nucleotide”, “oligonucleotide”, and “polynucleotide” are intended to include those moieties that contain the natural nucleotides (A, T, G, C, U), as well as those moieties that contain modified nucleotides, such as those in which the purine and pyrimidine bases have been modified or replaced with other heterocyclic bases. Such modifications include methylated purines or pyrimidines, acylated purines or pyrimidines, alkylated riboses or other heterocycles. In addition, the terms “nucleoside”, “nucleotide”, “oligonucleotide”, and “polynucleotide” include those moieties that contain not only conventional ribose and deoxyribose sugars, but other sugars as well. Modified nucleosides or nucleotides may have modifications on the sugar moiety, e.g., wherein one or more of the hydroxyl groups are replaced with halogen atoms or aliphatic groups, or are functionalized as ethers, amines, or the like. Modified nucleosides or nucleotides also include molecules having structural features that are recognized in the literature as being mimetics, derivatives, having similar properties, or other like terms, and include, for example, polynucleotides incorporating non-natural (not usually occurring in nature) nucleotides, non-natural nucleotide mimetics such as 2′-modified nucleosides, peptide nucleic acids (PNA), locked nucleoside analogues (LNA), oligomeric nucleoside phosphonates, and any polynucleotide that has added substituent groups, such as protecting groups or linking moieties.

An “add-in oligo” is an oligonucleotide that is included in a hybridization assay and is intended to bind to an add-in oligo complementary region of a probe, as described herein. Thus, the sequence of both the add-in oligo and the add-in oligo complementary region of the probe are generally known in advance of the hybridization assay. An add-in oligo typically does not originate from the sample; in other words, the add-in oligo is not typically isolated from source of the sample and is distinct from the sample. The add-in oligo is typically supplied as a reagent for use in the methods of the present invention, and may be mixed with the sample during a hybridization assay as further described herein. An “add-in oligo complementary region” is generally a portion of a probe (e.g. on an array) that is intended to bind to an add-in oligo that is included in a hybridization assay. The add-in oligo is typically about 8 to about 40 nucleotides long, e.g. about 10 to about 25 nucleotides long, or about 12 to about 20 nucleotides long. In certain embodiments, the add-in oligo may be about 8 to about 75 nucleotides long. The add-in oligo complementary region is typically about 8 to about 40 nucleotides long, e.g. about 10 to about 25 nucleotides long, or about 12 to about 20 nucleotides long. In certain embodiments, the add-in oligo complementary region may be about 8 to about 75 nucleotides long. Either or both of the add-in oligo and the add-in oligo complementary region may have sequences that include one or more modified nucleotides, such as modified oligonucleotides described above or as otherwise known in the literature. In particular embodiments in accordance with the present invention, either or both of the add-in oligo and the add-in oligo complementary region are made up of the nucleotides typically found naturally in living organisms or those typically used in chemical synthesis of oligonucleotides (e.g. individual nucleotides typically referred to by the abbreviations A, T, G, C, and U in the literature). In typical embodiments, the add-in oligo is made up of ribonucleotides, e.g. the add-in oligo is a ribonucleic acid. In some embodiments, the add-in oligo is made up of deoxyribonucleotides, e.g. the add-in oligo is a deoxyribonucleic acid. In certain embodiments, the add-in oligo may include both ribonucleotides and deoxyribonucleotides; i.e. a single molecule of an add-in oligo may have an RNA sequence joined to a DNA sequence. In certain embodiments, the add-in oligo is a polynucleotide incorporating at least one non-natural nucleotide. In typical embodiments, an add-in oligo is designed so that it does not specifically bind directly to any of the target complementary regions present on the array. For example, the sequence of the add-in oligo is not complementary to any target complementary regions present on the array. Also, in typical embodiments, the add-in oligo complementary region is designed so that it does not specifically bind directly to any known target analyte (e.g. small RNA or other component of the sample containing the small RNA) that the probes of the array are directed to. For example, the sequence of the add-in oligo complementary region is not complementary to any known target analyte that the probes of the array are directed to.

A “target complementary region” is generally a portion of a probe (e.g. on an array) that is intended to bind to a target during the hybridization assay, for example a target small RNA. The target complementary region generally contains a contiguous nucleotide sequence that is complementary to the nucleotide sequence of a corresponding target small RNA (e.g. target miRNA) and is of a length that is sufficient to provide specific binding between the probe and the corresponding small RNA. Since miRNAs are generally in the range of about 19 to about 25 nucleotides (nt) in length, in certain embodiments the target complementary region is generally at least about 10 nt, at least about 12 nt, or at least about 15 nt in length. In certain embodiments target complementary region may be as long as about 18 nt, as long as about 20 nt, as long as about 22 nt, or as long as about 25 nt in length, or longer. In certain embodiments, the target complementary region may be as long as about 30 nt, as long as about 40 nt, as long as about 50 nt, or longer. The target complementary region therefore is directed to (e.g. hybridizes to and may be used to detect) a particular target small RNA.

“Sequence” may refer to a particular sequence of bases and/or may also refer to a polynucleotide having the particular sequence of bases. Thus a sequence may be information or may refer to a molecular entity, as indicated by the context of the usage. A duplex is a double stranded structure typically formed between complementary nucleic acid sequences. An intermolecular duplex is a double stranded structure typically formed between two different polynucleotide molecules that have complementary nucleic acid sequences, wherein the complementary nucleic acid sequences are hybridized to each other.

“Moiety” and “group” are used to refer to a portion of a molecule, typically having a particular functional or structural feature, e.g. a linking group (a portion of a molecule connecting two other portions of the molecule), or an ethyl moiety (a portion of a molecule with a structure closely related to ethane). A moiety is generally bound to one or more other moieties to provide a molecular entity. As a simple example, a hydroxyl moiety bound to an ethyl moiety provides an ethanol molecule. At various points herein, the text may refer to a moiety by the name of the most closely related structure (e.g. an oligonucleotide moiety may be referenced as an oligonucleotide, a mononucleotide moiety may be referenced as a mononucleotide). However, despite this seeming informality of terminology, the appropriate meaning will be clear to those of ordinary skill in the art given the context, e.g. if the referenced term has a portion of its structure replaced with another group, then the referenced term is usually understood to be the moiety. For example, a mononucleotide moiety is a single nucleotide which has a portion of its structure (e.g. a hydrogen atom, hydroxyl group, or other group) replaced by a different moiety (e.g. a linking group, an observable label moiety, or other group). Similarly, an oligonucleotide moiety is an oligonucleotide which has a portion of its structure (e.g. a hydrogen atom, hydroxyl group, or other group) replaced by a different moiety (e.g. a linking group, an observable label moiety, or other group). “Nucleotide moiety” is generic to both mononucleotide moiety and oligonucleotide moiety.

“Bound” may be used herein to indicate direct or indirect attachment. In the context of chemical structures, “bound” (or “bonded”) may refer to the existence of a chemical bond directly joining two moieties or indirectly joining two moieties (e.g. via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect. “Free,” as used in the context of a moiety that is free, indicates that the moiety is available to react with or be contacted by other components of the solution in which the moiety is a part. Two moieties “directly bound” to each other are joined to each other without any intervening moiety.

“Isolated” or “purified” generally refers to isolation of a substance (compound, polynucleotide, protein, polypeptide, polypeptide, chromosome, etc.) such that the substance comprises a substantial portion of the sample in which it resides (excluding solvents), i.e. greater than the substance is typically found in its natural or un-isolated state. Typically, a substantial portion of the sample comprises at least about 1%, at least about 2%, at least about 5%, at least about 10%, at least about 20%, at least about 30%, at least about 50%, at least about 80%, or at least about 90% of the sample (excluding solvents). For example, a sample of isolated RNA (an “isolated RNA sample”) typically refers to a sample of RNA obtained using an RNA purification protocol on a starting mixture that include the RNA desired to be purified. An “isolated RNA sample” typically comprises at least about 2% total RNA, or at least about 5% total RNA, where percent is calculated in this context as mass (e.g. in micrograms) of total RNA in the sample divided by mass (e.g. in micrograms) of the sum of (total RNA+other constituents in the sample (excluding solvent)). Techniques for purifying polynucleotides and polypeptides of interest are well known in the art and include, for example, gel electrophoresis, ion-exchange chromatography, affinity chromatography, and sedimentation according to density.

The term “sample” as used herein relates to a material or mixture of materials, typically, although not necessarily, in fluid form, containing one or more components of interest.

The term “analyte” is used herein to refer to a known or unknown component of a sample. In certain embodiments of the invention, an analyte may specifically bind to a capture agent on a support surface if the analyte and the capture agent are members of a specific binding pair. In general, analytes are typically RNA or other polynucleotides. Typically, an “analyte” is referenced as a species in a mobile phase (e.g., fluid), to be detected by a “capture agent” which, in some embodiments, is bound to a support, or in other embodiments, is in solution. However, either of the “analyte” or “capture agent” may be the one which is to be evaluated by the other (thus, either one could be an unknown mixture of components of a sample, e.g., polynucleotides, to be evaluated by binding with the other). A “target” references an analyte.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides, or fragments of double stranded or single-stranded DNA or RNA may employed. Capture agents usually “specifically bind” one or more analytes.

The terms “specific binding”, “specifically bind”, or other like terms, refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the binding constant of a capture agent and analyte is greater than 10⁶ M⁻¹, greater than 10⁷ M⁻¹, greater than 10⁸ M⁻¹, greater than 10⁹ M⁻¹, greater than 10¹⁰ M⁻¹, usually up to about 10¹² M⁻¹, or even up to about 10¹⁵ M⁻¹. “Specific binding conditions” are conditions sufficient to allow a capture agent to preferentially bind to a particular analyte, e.g. stringent assay conditions.

The term “stringent assay conditions” as used herein refers to conditions that are compatible to produce binding pairs of nucleic acids, e.g., capture agents and analytes, of sufficient complementarity to provide for the desired level of specificity in the assay while being incompatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in Southern or Northern hybridizations, or hybridization of molecules in solution, or in array assays) are sequence dependent, and are different under different experimental conditions. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.1×SSC and 0.1% SDS at 37° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions may affect the degree to which nucleic acids are specifically hybridized to complementary capture agents. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 1 to about 20 minutes; or, multiple washes with a solution with a salt concentration of about 0.1×SSC containing 0.1% SDS at 20 to 50° C. for 1 to 15 minutes; or, equivalent conditions. Stringent conditions for washing can also be, e.g., 0.2×SSC/0.1% SDS at 42° C. In instances wherein the nucleic acid molecules are oligodeoxynucleotides (e.g. oligonucleotides made up of deoxyribonucleotide subunits), stringent conditions can include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides). See Sambrook, Ausubel, or Tijssen (cited below) for detailed descriptions of equivalent hybridization and wash conditions and for reagents and buffers, e.g., SSC buffers and equivalent reagents and conditions.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “array” and the equivalent term “microarray” each reference an ordered array of capture agents for binding to aqueous analytes and the like. An “array” includes any two-dimensional or substantially two-dimensional (as well as a three-dimensional) arrangement of spatially addressable regions (i.e., “features”) containing capture agents, particularly polynucleotides, and the like. In this regard, “a probe” references a capture agent that is a member of a set of probes as described herein. Any given support may carry one, two, four or more arrays disposed on a surface of a support. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 100 cm², 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Arrays can be fabricated by depositing (e.g., by contact- or jet-based methods) either precursor units (such as nucleotide or amino acid monomers) or pre-synthesized capture agent. An array is “addressable” when it has multiple regions of different moieties (e.g., different capture agents) such that a region (i.e., a “feature” or “spot” of the array) at a particular predetermined location (i.e., an “address”) on the array will detect a particular target. An “array layout” refers to one or more characteristics of the features, such as feature positioning on the array support, one or more feature dimensions, and an indication of a moiety at a given location. Capture agents on the array are typically selected based on the sequences of the intended target analytes. Particular capture agents or strategies for designing capture agents are described in copending U.S. patent application Ser. No. 11/173,693 filed on Jul. 1, 2005 by Wang entitled “Nucleic Acid Probes for Analysis of Small RNAs and Other Polynucleotides”; the copending U.S. patent application Ser. No. 11/256,229 filed on Oct. 21, 2005 by Wang et al. entitled “Analysis of microRNA” and designated attorney docket no. 10051514-1; and the copending U.S. patent application Ser. No. 11/264,788 filed on Oct. 31, 2005 by Wang entitled “Probe/Target Stabilization with Add-In Oligo” and designated attorney docket no. 10051644-1. “Interrogating” the array refers to obtaining information from the array, especially information about analytes binding to the array. “Hybridization assay” references a process of contacting an array with a mobile phase containing analyte. An “array support” refers to an article that supports an addressable collection of capture agents, and may be, e.g. an insoluble support, a planar support, or any other kind of support known in the microarray art. In particular embodiments, the support may be a substrate made of glass (e.g. a slide), plastic, metal, or other available material compatible with manufacture and use of the array.

The term “predetermined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e., a known analyte that is of interest, is used synonymously with the term “pre-determined analyte”.

Small RNA references RNAs less than about 300 bases long, generally less than about 200 bases long, e.g. less than about 100 bases long, less than about 60 bases long, less than about 50 bases long, less than about 40 bases long, or less than about 35 bases long. In particular embodiments, the small RNA may be selected from short interfering RNAs (siRNAs), microRNAs (miRNA), tiny non-coding RNAs (tncRNA) and small modulatory RNA (smRNA), or combinations thereof. See Novina et al., Nature 430: 161-164 (2004). In particular embodiments, small RNAs may be at least about 4 bases long, at least about 6 bases long, at least about 8 bases long, or longer.

Long polynucleotide references a polynucleotide that is separable from small RNAs using a size fractionation method as described herein or as known in the art to provide a fraction containing small RNA and another fraction containing other polynucleotides having lengths generally longer than about 300 bases. Long polynucleotides are polynucleotides generally longer than about 300 bases, e.g. longer than about 400 bases, longer than about 500 bases, longer than about 700 bases, and may be up to about 5000 bases long, up to about 10,000 bases long, or even longer.

“Complementary” references a property of specific binding between polynucleotides based on the sequences of the polynucleotides. As used herein, polynucleotides are complementary if they bind to each other in a hybridization assay under stringent conditions, e.g. if they produce a given or detectable level of signal in a hybridization assay. Portions of polynucleotides are complementary to each other if they follow conventional base-pairing rules, e.g. A pairs with T (or U) and G pairs with C. “Complementary” includes embodiments in which two polynucleotides are strictly complementary and also includes embodiments in which two polynucleotides are substantially complementary. In this regard, “strictly complementary” is a term used to characterize a first polynucleotide and a second polynucleotide, such as a target and a capture agent directed to the target, and means that every base in a sequence (or sub-sequence) of contiguous bases in the first polynucleotide has a corresponding complementary base in a corresponding sequence (or sub-sequence) of contiguous bases in the second polynucleotide. “Strictly complementary” means that there are no insertions, deletions, or substitutions in either of the first and second polynucleotides with respect to the other polynucleotide (over the complementary region). Put another way, every base of the complementary region may be paired with its complementary base, e.g. following normal base-pairing rules. “Substantially complementary” is a term used to characterize a first polynucleotide and a second polynucleotide, and means that there may be one or more relatively small (less than 10 bases, e.g. less than 5 bases, typically less than 3 bases, more typically a single base) insertions, deletions, or substitutions in the first and/or second polynucleotide (over the complementary region) relative to the other polynucleotide. The complementary region is the region that is complementary between a first polynucleotide and a second polynucleotide (e.g. a target analyte and a capture agent; further e.g. a small RNA and a small RNA binding site in a long polynucleotide such as a messenger RNA). Complementary sequences are typically embedded within larger polynucleotides, thus two relatively long polynucleotides may be complementary over only a portion of their total length. The complementary region is typically at least about 10 bases long, more typically at least about 12 bases long, more typically at least about 15 bases long, still more typically at least about 20 bases long, or may be at least about 25 bases long. In various typical embodiments, the complementary region may be up to about 200 bases long, or up to about 120 bases long, up to about 100 bases long, up to about 80 bases long, up to about 60 bases long, up to about 45 bases long, or up to about 40 bases long.

If a polynucleotide, e.g. a capture agent, is “directed to” a target, the polynucleotide has a sequence that is complementary to a sequence in that target and will specifically bind (e.g. hybridize) to that target under hybridization conditions. The hybridization conditions typically are selected to produce binding pairs of nucleic acids, e.g., capture agents and targets, of sufficient complementarity to provide for the desired level of specificity in the assay while being incompatible to the formation of binding pairs between binding members of insufficient complementarity to provide for the desired specificity. Such hybridization conditions are typically known in the art. Examples of such appropriate hybridization conditions are also disclosed herein for hybridization of a sample to an array. The target will typically be a small RNA, e.g. an miRNA, for embodiments discussed herein.

Accordingly, the invention thus relates to novel methods of performing an array analysis of an RNA sample. In certain embodiments, the invention provides a method of analyzing a sample containing small RNAs. The sample is contacted with an array in the presence of an add-in oligo under conditions sufficient to provide for binding to the array. The array has a set of probes bound to an array support. Each probe of the set has a target complementary region bound to the array support and an add-in oligo complementary region bound to the target complementary region. Thus, the add-in oligo complementary region is bound to the array support via the target complementary region. The target complementary region of each probe of the set is directed to a small RNA of interest. The array is then interrogated to obtain information about small RNAs in the sample. The present invention also relates to arrays such as those used in the above-described method. Kits in accordance with the present invention are also described.

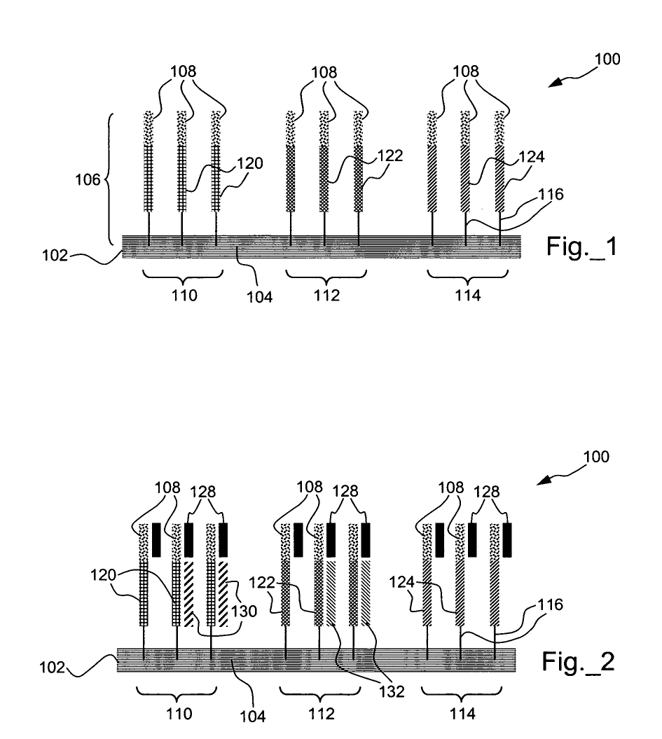

Referring now to FIG. 1, an embodiment of an array 100 in accordance with the invention is illustrated. The array 100 includes an array support 102 having a surface 104. Probes 106 are bound to the surface 104 of the array support 102 to provide features 110, 112, 114 of the array. The probes 106 make up a set of probes bound to the surface 104. Each of the probes 106 includes a target complementary region 120, 122, 124 and, optionally, a linker moiety 116. The target complementary region 120, 122, 124 is typically bound to the surface 104 via the linker moiety 116, as shown in FIG. 1. The linker moiety 116 is optional; thus, in certain embodiments, the target complementary region 120, 122, 124 is bound to the surface 104 directly. Each probe 106 further includes an add-in oligo complementary region 108 bound directly to the target complementary region 120, 122, 124. Thus, the add-in oligo complementary region 108 is bound to the surface 104 via the target complementary region 120, 122, 124 and the optional linker moiety (if present). The linker moiety, if present, is typically a polymer that does not interact or hybridize with the target small RNAs or the add-in oligo. A suitable linker moiety may be, for example, about 5 to about 20 nucleotides long.

The add-in oligo complementary region 108 generally has the same sequence in all of the probes 106 of the array 100. The probes 106 of a given feature 110, 112, 114 typically all have the same target complementary region. The identity (sequence) of a target complementary region will typically differ from feature to feature of the array, indicated in FIG. 1 by having three different target complementary regions 120, 122, 124, each target complementary region 120, 122, 124 corresponding to a different feature 110, 112, 114 of the array 100. Each of the target complementary regions is directed to a small RNA and is capable of binding to its respective small RNA during a hybridization assay, e.g. when a sample containing small RNAs is contacted with the array under conditions sufficient to provide for specific binding, e.g. under stringent hybridization conditions. In certain embodiments the array includes a set of probes which is made up of a plurality of sub-sets of probes, each subset corresponding to a different feature 110, 112, 114, wherein every probe in a given subset has the same target complementary sequence, and different subsets of probes have different target complementary sequences. In certain embodiments, the array includes other capture agents in addition to set of probes; such other capture agents may be directed to control polynucleotides, analytes other than small RNAs, other transcripts, etc.

In an embodiment in accordance with the present invention, an array 100 such as that shown in FIG. 1 is employed in a method of performing an array analysis of an RNA sample, e.g. a sample that includes small RNAs. In exemplary methods described herein, a sample containing small RNAs is contacted with the array 100 in the presence of an add-in oligo 128 under conditions sufficient to provide for binding to the array. FIG. 2 shows an array 100 which has been contacted with a sample of small RNAs. The add-in oligo 128 is hybridized (bound) to the add-in oligo complementary region 108 (forming a duplex). Target complementary regions 120 and 122 are hybridized to small RNAs 130 and 132 from the sample (again, forming duplexes). The probes disposed at the feature 110 specifically bind to small RNA 130, and the probes disposed at the feature 112 specifically bind to a different small RNA 132. Feature 114 illustrates that the array 100 may include some probes directed to small RNAs that are not present in the sample. Thus, in particular embodiments, lack of binding at a feature 114 (e.g. lack of a signal at the feature 114 during interrogation) provides an indication that the sample analyzed did not include the small RNA corresponding to the target complementary region 124 of the probes at feature 114.

The sequence of the target complementary regions 120, 122, 124 are selected during the probe design process to be capable of base-pairing (e.g. during a hybridization assay) to a target small RNA. As shown in FIG. 2, the terminal-most nucleotides of the target small RNA 130, 132 are capable of base-pairing with the terminal-most nucleotides of the target complementary region 120, 122 adjacent the add-in oligo complementary region 108. Similarly, the terminal-most nucleotides of the add-in oligo complementary region 108 adjacent the target complementary region 120, 122, 124 are capable of base-pairing with the terminal-most nucleotides of the add-in oligo 128. “Terminal-most nucleotides” references the nucleotides at an end (e.g. the 3′ or 5′ end) of a polynucleotide or at an end (e.g. the 3′ or 5′ end) of an indicated region of a probe (e.g. the target complementary region or the add-in oligo complementary region); the “terminal-most nucleotides” generally includes at least 2 (e.g. at least 3, at least 4, at least 5, or more) contiguous nucleotides starting with the terminal nucleotide.

In typical embodiments, the terminal-most nucleotides of the target small RNA 130, 132 are strictly complementary to the terminal-most nucleotides of the target complementary region 120, 122 adjacent the add-in oligo complementary region 108. Similarly, in some typical embodiments, the terminal-most nucleotides of the add-in oligo complementary region 108 adjacent the target complementary region 120, 122 are strictly complementary to the terminal-most nucleotides of the add-in oligo 128.

As noted above, the target complementary region 120, 122, 124 is directly bound to the add-in oligo complementary region 108. Thus, as illustrated in FIG. 2, when a small RNA 130, 132 and an add-in oligo 128 are both bound to a given probe, an end of the small RNA 130, 132 is directly adjacent an end of the add-in oligo 128. Without being bound to any single theory of operation of the invention, it is believed that this orientation (with an end of the small RNA adjacent an end of the add-in oligo) provides for base-stacking to occur between the terminal nucleotide of the add-in oligo and the directly adjacent terminal nucleotide of the small RNA. This base-stacking stabilizes binding of the small RNA. Also, the add-in oligo may serve to destabilize or obstruct binding by any polynucleotide species (e.g. pre-miRNA, pri-miRNA, messenger RNA, other long transcripts) having a sequence similar to the small RNA but which are longer and have additional sequence beyond normal end of the small RNA.

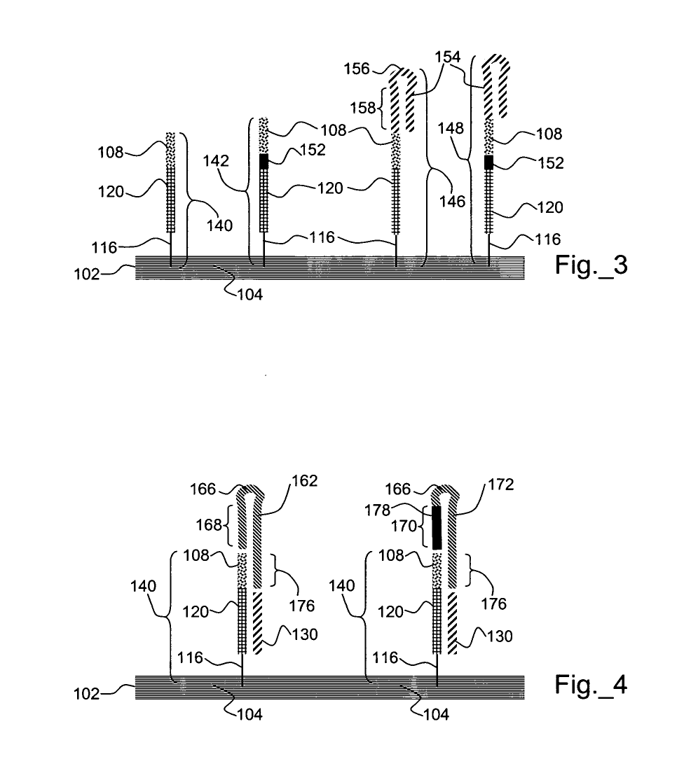

It is contemplated that the probes may include further regions, such as the Tm enhancement domains described in U.S. patent application Ser. No. 11/173,693, cited at the beginning of this specification. FIG. 3 illustrates such probes 140, 142, 146, 148 bound to a surface 104 of an array support 102. Probe 140 is a probe, such as described above, having a target complementary region 120 and, optionally, a linker moiety 116. The target complementary region 120 is typically bound to the surface 104 via the linker moiety 116. The linker moiety 116 is optional; thus, in certain embodiments, the target complementary region 120 is bound to the surface 104 directly. The probe 140 further includes an add-in oligo complementary region 108 bound to the target complementary region 120. Thus, the add-in oligo complementary region 108 is bound to the surface 104 via the target complementary region 120 and the optional linker moiety (if present). In particular embodiments, the probe is about 20 to about 150 nucleotides long, typically about 25 to about 100 nucleotides long, more typically about 30 to about 80 nucleotides long. The probe 140 may be attached via its 3′ end or its 5′ end to the array support 102.

Also shown in FIG. 3 is a probe 142 that includes the add-in oligo complementary region 108, optional linker moiety 116, and target complementary region 120, similar to that described for probe 140. In addition, probe 142 includes a nucleotide clamp 152. One end of nucleotide clamp 152 is bound directly to the target complementary region 120 and another end of nucleotide clamp 152 is bound directly to the add-in oligo complementary region 108. Thus, the add-in oligo complementary region 108 is bound to the surface 104 via the nucleotide clamp 152, the target complementary region 120, and the optional linker moiety 116 (if present), in that order. The nucleotide clamp 152 contains a contiguous sequence of up to about 5 nucleotides (i.e., 1, 2, 3, 4 or 5 nucleotides), wherein the identity of the nucleotides employed in the nucleotide clamp may be the same as each other or different from each other. The nucleotide clamp 152 typically contains nucleotides selected from G and C, possibly A, T, or U, or a modified nucleotide.

Also shown in FIG. 3 is a probe 146 that includes the add-in oligo complementary region 108, target complementary region 120, and optional linker moiety 116 as described for probe 140. In addition, probe 146 includes a hairpin structure 154 bound directly to the add-in oligo complementary region 108. Thus, the hairpin structure 154 is bound to the surface 104 via the add-in oligo complementary region 108, the target complementary region 120, and the optional linker moiety 116 (if present), in that order. The hairpin structure 154 typically has a loop 156 of at least 3 or 4 nucleotides (typically up to about 8 or 10 nucleotides) and a double-stranded stem 158 (of about 6 to about 20 base pairs) in which complementary nucleotides bind to each other in an anti-parallel manner.

Also shown in FIG. 3 is a probe 148 that includes the add-in oligo complementary region 108, nucleotide clamp 152, target complementary region 120, and optional linker moiety 116 as described for probe 142. In addition, probe 148 includes a hairpin structure 154 bound directly to the add-in oligo complementary region 108. Thus, the hairpin structure 154 is bound to the surface 104 via the add-in oligo complementary region 108, the nucleotide clamp 152, the target complementary region 120, and the optional linker moiety 116 (if present), in that order.

In some embodiments, the add-in oligo may include a hairpin structure. One such embodiment is illustrated in FIG. 4. In FIG. 4 probes 140 are bound to a surface 104 of an array support 102. Probe 140 is a probe such as described above having an add-in oligo complementary region 108, a target complementary region 120 and, optionally, a linker moiety 116. Target complementary regions 120 are hybridized to small RNAs 130 from the sample (forming duplexes). FIG. 4 illustrates two different embodiments 162, 172 of an add-in oligo having a hairpin structure. Each of the add-in oligos 162, 172 include a sequence 176 that is capable of specifically binding to the add-in oligo complementary region 108 (forming a duplex). Each of the add-in oligos 162, 172 further includes a hairpin structure that typically has a loop 166 of at least 3 or 4 nucleotides (typically up to about 8 or 10 nucleotides) and a double-stranded stem 168, 170 (of about 6 to about 20 base pairs) in which complementary nucleotides bind to each other in an anti-parallel manner. In typical embodiments, the add-in oligo is composed of ribonucleotides, e.g. the add-in oligo is a ribonucleic acid. In some embodiments, the add-in oligo is composed of deoxyribonucleotides, e.g. the add-in oligo is a deoxyribonucleic acid. The nucleotides of the stem 168, 170, the loop 166, and sequence 176 may, in various embodiments, be independently selected from deoxyribonucleotides, ribonucleotides, and modified nucleotides. The embodiment of add-in oligo 162 is composed of nucleotides that are all the same type, e.g. the nucleotides are all ribonucleotides or are all deoxyribonucleotides. The embodiment of add-in oligo 172 is composed of nucleotides that are of different types, e.g. the add-in oligo 172 includes both ribonucleotides and deoxyribonucleotides; e.g. the add-in oligo may, in a single molecule, have an RNA sequence joined to a DNA sequence. In one possible embodiment, add-in oligo 172 includes a DNA sequence 178 joined to an RNA sequence (the remainder of add-in oligo 172).

The subject invention provides methods of analyzing a sample for small RNA, e.g. assessing for the presence or amount of a miRNA. In general, the subject methods include: a) contacting an array with the sample in the presence of an add-in oligo; and b) interrogating the array to obtain information about small RNA in the sample. The array has a set of probes bound to an array support. Each probe of the set of probes has a target complementary region bound to the array support and an add-in oligo complementary region bound to the target complementary region, wherein the add-in oligo complementary region is bound to the array support via the target complementary region. In certain embodiments, the add-in oligo complementary region is directly bound to the target complementary region; in other embodiments, the add-in oligo complementary region is directly bound to a nucleotide clamp, and the nucleotide clamp is directly bound to the target complementary region. The target complementary region of each probe of the set is directed to a small RNA of interest. The array is typically contacted with the sample comprising small RNA under specific binding conditions, e.g. stringent assay conditions. Interrogating the array typically involves detecting the presence of any detectable label associated with the probes, thereby evaluating the amount of the respective targets, e.g. small RNAs such as miRNAs, in the sample.

The sample of RNA may be obtained from any source capable of providing RNA. For example, the sample of RNA may be any RNA sample, typically a sample containing RNA that has been isolated from a biological source, e.g. any plant, animal, yeast, bacterial, or viral source, or a non-biological source, e.g. chemically synthesized. The sample may already be in solution form or may be a dried sample of RNA to which a reconstitution buffer is added. In particular embodiments, the sample of RNA includes one or more small RNAs, such as e.g. short interfering RNAs (siRNAs), microRNAs (miRNA), tiny non-coding RNAs (tncRNA) and small modulatory RNA (smRNA). See Novina et al., Nature (2004) 430: 161-164. In particular embodiments, the sample includes isolated small RNAs, e.g. the sample results from an isolation protocol for small RNA such as one or more of those listed in this paragraph. In certain embodiments, the small RNA targets may include isolated miRNAs, such as those described in the literature and in the public database accessible via the at the world-wide website of the Sanger Institute (Cambridge, UK) (which may be accessed by typing “www” followed by “.sanger.ac.uk/cgi-bin/Rfam/mima/browse.pl” into the address bar of a typical internet browser). Methods for preparing samples of miRNAs from cells are well known in the art (see, e.g., Lagos-Quintana et al, Science 294:853-858(2001); Grad et al, Mol Cell 11:1253-1263 (2003); Mourelatos et al, Genes Dev 16:720-728(2002); Lagos-Quintana et al, Curr Biol 12:735-739(2002); Lagos-Quintana et al, RNA 9:175-179(2003) and other references cited above). In some embodiments, the sample of RNA may be a whole RNA fraction isolated from a biological source and includes messenger RNA and small RNA. Such samples including a diverse set of RNAs, such as a whole RNA fraction, may be referenced herein as “complex” RNA samples.

In particular embodiments of a method in accordance with the present invention, the method includes obtaining an initial mixture containing RNA and separating components in the initial mixture based on the molecular size of the components. In certain embodiments, the method includes isolating small RNAs, especially RNAs less than about 300 bases long, e.g. less than about 200 bases long, less than about 100 bases long, or less than about 50 bases long. The size-fractionation of the initial mixture containing RNA thus provides an isolated RNA sample that includes isolated small RNAs, e.g. RNAs less than about 300 bases long (e.g. less than about 200 bases long, less than about 100 bases long, less than about 50 bases long). Any fractionation method capable of providing the isolated RNA sample may be employed. Typical methods of fractionating mixtures of polynucleotides according to size are known and need not be described in detail here. In particular embodiments, a size-based separation of the sample is performed by contacting the sample with a size fractionation medium under denaturing conditions and recovering a fraction containing the small RNAs that are of interest; such a method is described in a U.S. patent application Ser. No. 11/264,783 filed on Oct. 31, 2005 by Wang entitled “Denaturing Size-Fractionation in Analysis of small RNA” and assigned attorney docket number 10050949-1.

In various embodiments, at least about 10% (e.g. at least about 20%, 40% or 60%) of the RNAs in the sample comprising the small RNAs are shorter than about 300 bases, e.g. generally shorter than about 200 bases, shorter than about 100 bases. This percentage is calculated as: (mass of RNAs less than about 300 (or less than about 200, or less than about 100) bases long in a given volume of the sample comprising the small RNAs) divided by (total mass of RNA in the given volume of the sample comprising the small RNAs), and then expressed as a percentage.

In certain embodiments, long polynucleotides constitute less than about 80% (e.g. less than about 60%, less than about 40%, less than about 20%, less than about 10%) of the total polynucleotides in the sample comprising the small RNAs. This percentage is calculated as: (mass of long polynucleotides in a given volume of the sample comprising the small RNAs) divided by (total mass of polynucleotides in the given volume of the sample comprising the small RNAs), and then expressed as a percentage. In certain embodiments, long polynucleotides (e.g. polynucleotides longer than about 300 bases, longer than about 400 bases, longer than about 500 bases) make up an insubstantial amount of the sample comprising the small RNAs. In this regard, “an insubstantial amount” is an amount which does not substantially interfere with binding of small RNAs to the probes of the array, i.e. less than about 5% error is introduced into the binding measurements obtained during interrogation of the array due to the presence of the long polynucleotides.

In particular embodiments, the small RNAs in the sample are labeled prior to being contacted with the array. In certain embodiments, the sample may be isolated from a source already labeled. In typical embodiments, binding of labeled small RNAs to the probes of the array is detected by detecting the label associated with the probes (due to binding of the labeled small RNAs). In general, labeling methods are well known in the art (e.g., using RNA ligase, polyA polymerase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.), and, accordingly, such methods do not need to be described here in great detail. The observable label may be any observable label known in the art, e.g. a chromophore, a fluorescent label, a spin label, a radioisotope label, a mass label, a sequence label, a chemically reactive tag, an affinity label, or any other known label. In particular embodiments, the label is a fluorescent dye, which labels will be described in greater detail below.

Fluorescent dyes of particular interest include: xanthene dyes, e.g. fluorescein and rhodamine dyes, such as fluorescein isothiocyanate (FITC), 6 carboxyfluorescein (commonly known by the abbreviations FAM and F), 6 carboxy-2′,4′,7′,4,7-hexachlorofluorescein(HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarins, e.g. umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc. More information about commercially available dyes for oligonucleotide conjugation can be found at the Synthegen website (which may be accessed by typing “www” followed by “.synthegen.com” into the address bar of a typical internet browser). Any such dyes may potentially be used in accordance with the methods described herein. Such labels typically are well known in the art.

In certain embodiments, binding of labeled small RNAs is assessed with respect to binding of at least one labeled control sample. In one example, a suitable labeled control sample may be made from a control cell population. In certain embodiments, a sample and a control sample may be prepared and labeled, and relative binding of the labeled small RNAs from the samples to probes on an array may be assessed. Typically, the labeled small RNAs are contacted with the array under stringent hybridization conditions.

In practicing the subject methods, the sample and control sample may be labeled to provide at least two different populations of labeled small RNAs that are to be compared. The populations of small RNAs may be labeled with the same label or different labels, depending on the actual assay protocol employed. For example, where each population is to be contacted with different but identical arrays, each population of small RNAs may be labeled with the same label. Alternatively, where both populations are to be simultaneously contacted with a single array of surface-bound probes, i.e., co-hybridized to the same array of immobilized probes, the two different populations are generally distinguishably labeled with respect to each other.

The samples are sometimes labeled using “distinguishable” labels in that the labels that can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY VI 005 (Molecular Probes, Eugene, Oreg.), POPO-3 and TOTO-3 (Molecular Probes, Eugene, Oreg.), fluorescein and Texas red (Dupont, Boston Mass.) and POPRO3 and TOPRO3 (Molecular Probes, Eugene, Oreg.). Further suitable distinguishable detectable labels may be described in Kricka et al. (Ann. Clin. Biochem. 39:114-29, 2002).

In certain embodiments, at least a first population of small RNAs and a second population of small RNAs are produced from two different small RNA-containing samples, e.g., two populations of cells. As indicated above, depending on the particular assay protocol (e.g., whether both populations are to be hybridized simultaneously to a single array or whether each population is to be hybridized to two different but substantially identical, if not identical, arrays) the populations may be labeled with the same or different labels. As such, a feature of certain embodiments is that the different populations of small RNAs are labeled with the same label such that they are not distinguishably labeled. In yet other embodiments, a feature of certain embodiments is that the different populations of small RNAs are labeled with different labels such that they are distinguishable from each other.

Accordingly, in typical embodiments the subject methods include a hybridization assay that typically includes the following: (1) providing an array having a set of probes as described herein disposed on an array support; (2) contacting a sample containing small RNAs (e.g. labeled small RNAs) with the array in the presence of an add-in oligo, under conditions sufficient to provide for specific binding, e.g. typically under stringent hybridization conditions; (3) washing the array to remove nucleic acids not bound to the array during the hybridization; and (4) detection of the hybridized small RNAs. The reagents used in each of these steps and their conditions for use may vary depending on the particular application.

The array includes an array support and a set of probes bound to the surface of the support. In particular embodiments, a set of probes includes at least five probes such as described above (“subject probes”), wherein all of said at least five probes have the same add-in oligo complementary region and each of said at least five probes has a different target complementary region. In some embodiments, a set of probes includes at least 10 subject probes, at least 20 subject probes, at least 50 subject probes, at least 100 subject probes, at least 200 subject probes, or more subject probes, such as up to 1000 subject probes, up to 2000 subject probes, or even more subject probes. In certain embodiments, all of the subject probes have the same add-in oligo complementary region, and each of the subject probes has different target complementary region. Each probe of the probe set may include a linker and/or Tm enhancement domain, as described above with regard to FIG. 3. In some embodiments, each probe may include one or more non-natural nucleotides (e.g. modified nucleotides, locked nucleoside analogues, or any other known non-natural nucleotides).

As indicated above, the sample is contacted with the array in the presence of an add-in oligo. The add-in oligo is typically included in the solution contacting the array surface (the “hybridization mixture”) in an amount in excess of the amount of probes present on the array. For example, a quantity of add-in oligo may be included in the hybridization mixture that is at least about 1.1× molar excess (e.g. at least about 1.5× molar excess, at least about 2× molar excess, at least about 3× molar excess, at least about 3× molar excess, at least about 4× molar excess, at least about 5× molar excess, at least about 10× molar excess) over the amount of probes present on the array. In certain embodiments, somewhat less of the add-in oligo may be included in the hybridization mixture, such as about 0.9× or about 1× relative to the amount of probes present on the array. The quantity of the add-in oligo usually need not exceed about 20× molar excess, but in certain embodiments, a greater quantity may be added as long as it does not interfere with the assay. In typical embodiments, the add-in oligo is included in a sufficient quantity to substantially saturate the add-in oligo complementary regions of the probes. In this regard, “substantially saturate” means that at least about 60%, e.g. at least about 70%, at least about 80%, at least about 90%, at least about 95%, of the add-in oligo complementary regions are bound to add-in oligo, forming duplexes. The add-in oligo may be added to the hybridization mixture in any effective manner, e.g. the sample may be mixed with add-in oligo and then brought into contact with the array, or the array may be pre-hybridized with the add-in oligo, or the add-in oligo may be releasably bound to the surface of the array prior to the hybridization reaction and then released upon contact with the hybridization mixture.

As indicated above, hybridization is carried out under suitable hybridization conditions, which may vary in stringency as desired; typical conditions are sufficient to produce probe/target complexes on an array surface between complementary binding members, e.g., between surface-bound probes and labeled complementary small RNAs, and also between the add-in oligo complementary region of probes and the add-in oligo. In certain embodiments, stringent hybridization conditions may be employed. Representative stringent hybridization conditions that may be employed in these embodiments are provided above.

In typical embodiments, after a labeling reaction to label small RNAs, the sample containing the small RNAs is contacted with an array in the presence of add-in oligo. The conditions employed during the hybridization are sufficient to result in hybridization of the small RNAs to the probes that are directed to the small RNAs, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C., for example.

The above hybridization step may include agitation of the array and the sample containing the labeled small RNAs, where the agitation may be accomplished using any convenient protocol, e.g., shaking, rotating, spinning, and the like.

Standard hybridization techniques (e.g. under conditions sufficient to provide for specific binding of small RNA, e.g. target miRNAs, to the probes on the array) are used for contacting the sample with the array. Suitable methods are described in many references (e.g., Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol. 7, pgs 43-65 (Plenum Press, New York 1985). See also U.S. Pat. Nos. 6,335,167; 6,197,501; 5,830,645; and 5,665,549; the disclosures of which are herein incorporated by reference. The hybridization is typically performed under stringent hybridization conditions, as described herein and as known in the art. Selection of appropriate conditions, including temperature, salt concentration, polynucleotide concentration, time(duration) of hybridization, stringency of washing conditions, and the like will depend on experimental design, including source of sample, identity of probes, degree of complementarity expected, etc., and may be determined as a matter of routine experimentation for those of ordinary skill in the art.

Following hybridization, the array is typically washed to remove unbound nucleic acids. Washing may be performed using any convenient washing protocol, where the washing conditions are typically stringent, as described above.

Following hybridization and washing, as described above, the hybridization of target analytes (e.g. small RNAs) to the probes is then detected using standard techniques of reading the array, i.e. the array is interrogated. Reading the resultant hybridized array may be accomplished by illuminating the array and reading the location and intensity of resulting fluorescence at each feature of the array to detect any binding complexes (e.g. probe/target analyte duplexes) on the surface of the array. For example, a scanner may be used for this purpose that is similar to the AGILENT MICROARRAY SCANNER available from Agilent Technologies, Palo Alto, Calif. Other suitable devices and methods are described in U.S. Pat. No. 6,756,202 and U.S. Pat. No. 6,406,849. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from interrogating the array may be raw results (such as fluorescence intensity readings for each feature in one or more color channels) or may be processed results (such as those obtained by subtracting a background measurement, or by rejecting a reading for a feature which is below a predetermined threshold, normalizing the results, calculating log ratios for the results, and/or forming conclusions based on the pattern read from the array (such as whether or not a particular target miRNA may have been present in the sample, or whether or not a pattern indicates a particular condition of an organism from which the sample came).

By “normalization” is meant that data corresponding to two populations of polynucleotides are globally normalized to each other, and/or normalized to data obtained from controls (e.g., internal controls produce data that are predicted to be equal in value in all of the data groups). Normalization generally involves multiplying each numerical value for one data group by a value that allows the direct comparison of those amounts to amounts in a second data group. Several normalization strategies have been described (Quackenbush et al, Nat. Genet. 32 Suppl:496-501, 2002, Bilban et al Curr Issues Mol. Biol. 4:57-64, 2002, Finkelstein et al, Plant Mol. Biol. 48(1-2):119-31, 2002, and Hegde et al, Biotechniques. 29:548-554, 2000). Specific examples of normalization suitable for use in the subject methods include linear normalization methods, non-linear normalization methods, e.g., using lowest local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained using a support-bound polynucleotide capture agent directed to a particular control polynucleotide, where the control polynucleotide is included in the hybridization at a known concentration, for example.

In certain embodiments, results from interrogating the array are used to assess the level of binding of the small RNAs from the sample to probes on the array. The term “level of binding” means any assessment of binding (e.g. a quantitative or qualitative, relative or absolute assessment), usually done, as is known in the art, by detecting signal (i.e., pixel brightness) from a label associated with the small RNA, e.g. the sample is labeled. The level of binding of labeled small RNA to probe is typically obtained by measuring the surface density of the bound label (or of a signal resulting from the label).

Accordingly, since the arrays used in the subject assays may contain probes for a plurality of different small RNAs, the presence of a plurality of different small RNAs in a sample may be assessed. The subject methods are therefore suitable for simultaneous assessment of a plurality of small RNAs in a sample.

In certain embodiments, a surface-bound probe may be assessed by evaluating its binding to two populations of small RNAs that are distinguishably labeled. In these embodiments, for a single surface-bound probe of interest, the results obtained from hybridization with a first population of labeled small RNAs may be compared to results obtained from hybridization with the second population of small RNAs, usually after normalization of the data. The results may be expressed using any convenient means, e.g., as a number or numerical ratio, etc.

Accordingly, in typical embodiments a sample containing small RNAs is labeled, e.g. with Cy5 or Cy3, and hybridized onto an array as follows: The sample containing the small RNA is desalted (e.g. with BioRad MICRO BIO-SPIN™-6 columns, as directed by BioRad instructions) to remove excess observable label remaining from the labeling reaction. The desalted sample containing the small RNA is added to solution containing water, carrier (25-mer DNA with random sequence), and the add-in oligo. The resulting solution is heated at about 100° C. for approximately 1 minute per 10 microliters of solution, and then immediately cooled on ice. The cooled solution is then added to hybridization buffer and mixed carefully. The final solution is then contacted with the array, e.g. in a SUREHYB™ hybridization chamber (Agilent Part Number:G2534A), and placed on the rotisserie of a hybridization oven overnight. The hybridization temperature is typically in the range from about 50° C. to about 65° C., or in the range from about 55° C. to about 60° C., although temperatures outside this range (e.g. in the range from about 30° C. to about 65° C., or in the range from about 45° C. to about 65° C.) may be used depending on the other experimental parameters, e.g. hybridization buffer composition and wash conditions. After the hybridization is complete, the array is washed thoroughly and dried with nitrogen as needed. The array is scanned (e.g. with an Agilent Scanner, Agilent Product Number: G2565BA). The data is then evaluated (e.g. using Agilent Feature Extraction Software, Agilent Product Number: G2567AA) for hybridization efficiency and specificity. Data may be further analyzed, e.g. using Spotfire software and Microsoft Excel.

Also provided by the subject invention are kits for practicing the subject methods, as described above. The subject kits include at least an array having a set of probes bound to an array support. Each probe of the set of probes has a target complementary region bound to the array support and an add-in oligo complementary region bound to the target complementary region. The add-in oligo complementary region is bound to the array support via the target complementary region. The target complementary region of each probe of the set is directed to a small RNA of interest. For example, a kit may include such an array and an add-in oligo, as described above. In certain embodiments the subject kits may also include reagents for isolating small RNA from a source (e.g. a tissue, cell, tissue lysate) to provide an isolated sample of small RNA. In some embodiments the subject kits optionally also include one or more constituents selected from: reagents for labeling RNA; reagents for contacting the sample of RNA with the array, e.g., control samples, buffers, wash solutions, etc. The various components of the kit may be present in separate containers or certain compatible components may be precombined into a single container, as desired.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a suitable material, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable material.

The practice of the present invention will employ, unless otherwise indicated, conventional techniques of synthetic organic chemistry, biochemistry, molecular biology, and the like, which are within the skill of the art. Such techniques are explained fully in the literature. Unless otherwise defined herein, all technical and scientific terms used herein have the same meaning as commonly understood by one of ordinary skill in the art to which the invention belongs. The description herein is put forth so as to provide those of ordinary skill in the art with a complete disclosure of the methods and compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. Standard temperature and pressure are defined as 20° C. and 1 atmosphere.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties, provided that, if there is a conflict in definitions, the definitions provided herein shall control.

What is claimed is:

1. A method of analyzing a sample comprising small RNAs, the method comprising:
contacting an array with the sample in the presence of an add-in oligo, the array comprising a set of probes and an array support, each probe comprising a target complementary region bound to the array support and an add-in oligo complementary region bound to the target complementary region, the add-in oligo complementary region bound to the array support via the target complementary region; and
interrogating the array to obtain information about small RNAs in the sample.

2. The method of claim 1, wherein the add-in oligo complementary region of every probe of the probe set has the same sequence.

3. The method of claim 1, wherein the add-in oligo has a sequence that is not complementary to the sequence of any target complementary region of probes of the probe set.

4. The method of claim 1, wherein said sample comprising small RNAs is an isolated small RNA sample.

5. The method of claim 4, wherein RNAs shorter than about 300 bases constitute at least 10% of the total RNAs in the isolated RNA sample.

6. The method of claim 4, wherein RNAs shorter than about 300 bases constitute at least 60% of the total RNAs in the isolated RNA sample.

7. The method of claim 1, wherein the small RNAs comprise microRNAs.

8. The method of claim 1, wherein the probe set comprises at least 20 different probes, each of the probes directed to a different small RNA.

9. The method of claim 1, wherein the probe set comprises at least 20 different probes, each of the probes directed to a different microRNA.

10. The method of claim 1, wherein the add-in oligo complementary region is directly bound to the target complementary region.

11. The method of claim 1, wherein the add-in oligo complementary region is directly bound to a nucleotide clamp and the nucleotide clamp is directly bound to the target complementary region.

12. The method of claim 1, said contacting done under conditions sufficient to provide for binding of the small RNAs to the probes.

13. The method of claim 12, the add-in oligo present in an amount of at least IX relative to the amount of probes present on the array.

14. The method of claim 1, wherein the add-in oligo is selected from a ribonucleic acid, a deoxyribonucleic acid, a polynucleotide incorporating at least one non-natural nucleotide, or a polynucleotide comprising a DNA sequence bound to a RNA sequence.

15. The method of claim 1, wherein the add-in oligo comprises one or more Locked Nucleoside Analogues (LNA).

16. The method of claim 1, wherein at least one of the probes of the set of probes comprises one or more Locked Nucleoside Analogues (LNA).

17. The method of claim 1, wherein the add-in oligo comprises one or more moieties selected from a ribonucleotide, a deoxyribonucleotide, a modified nucleotide, a non-natural nucleotide, a Locked Nucleoside Analogues (LNA), and combinations thereof.

18. The method of claim 1, wherein the add-in oligo comprises a hairpin structure.

19. The method of claim 1, further comprising, prior to contacting the sample with the array, labeling the small RNAs with an observable label.

20. The method of claim 1, wherein the small RNAs in the sample comprising the small RNAs have an observable label.

21. An array comprising a set of probes and an array support, each probe of the set of probes comprising a target complementary region bound to the array support and an add-in oligo complementary region bound to the target complementary region, the add-in oligo complementary region bound to the array support via the target complementary region.

22. The array of claim 21, wherein each probe of the set of probes further comprises a linker moiety bound to the array support, the target complementary region bound to the array support via the linker moiety.

23. The array of claim 21, wherein each probe of the set of probes further comprises a nucleotide clamp, a hairpin structure, or both.

24. The array of claim 21, wherein the target complementary region of each probe is about 10 to about 35 nucleotides long.

25. The array of claim 21, wherein the target complementary region of each probe is directed to a small RNA independently selected from the group consisting of a short interfering RNA (siRNA), microRNA (miRNA), tiny non-coding RNA (tncRNA), and small modulatory RNA (smRNA).

26. The array of claim 21, wherein the add-in oligo complementary region is directly bound to the target complementary region.

27. The array of claim 21, wherein the add-in oligo complementary region is directly bound to a nucleotide clamp and the nucleotide clamp is directly bound to the target complementary region.

28. A kit comprising:
an array comprising a set of probes and an array support, each probe of the set of probes comprising a target complementary region bound to the array support and an add-in oligo complementary region bound to the target complementary region, the add-in oligo complementary region bound to the array support via the target complementary region; and
an add-in oligo.
